SEQUENCE LISTING (20 sequences)

SEQ ID NO: 1 - DNA, 242 bp, Gallus gallus
agggacagcccccccccaaagcccccagggatgtaattacgtccctcccccgctagggcagcagcgagccgcccggggctccggtccggtccggcgctcccccgcatccccgagccggcagcgtgcggggacagcccgggcacggggaaggtggcacgggatcgctttcctctgaacgcttctcgctgctctttgagcctgcagacacctggggggatacggggaaaaaagctttaggctga

SEQ ID NO: 2 - DNA, 63 bp, Streptomyces sp.
tcgggagtagtgccccaactggggtaacctttgagttctctcagttgggggcgtagggtcgcc

SEQ ID NO: 3 - DNA, 63 bp, Streptomyces sp.
ggcgaccctacgcccccaactgagagaactcaaaggttaccccagttggggcactactcccga

SEQ ID NO: 4 - DNA, 63 bp, Streptomyces sp.
aagccgcggtgcgggtgccagggcgtgcccttgggctccccgggcgcgtactccacctcaccc

SEQ ID NO: 5 - DNA, 63 bp, Streptomyces sp.
gggtgaggtggagtacgcgcccggggagcccaagggcacgccctggcacccgcaccgcggctt

SEQ ID NO: 6 - DNA, 63 bp, Streptomyces sp.
aagccgcggtgcgggtgccagggcgtgcccttgagttctctcagttgggggcgtagggtcgcc

SEQ ID NO: 7 - DNA, 63 bp, Streptomyces sp.
ggcgaccctacgcccccaactgagagaactcaagggcacgccctggcacccgcaccgcggctt

SEQ ID NO: 8 - DNA, 63 bp, Streptomyces sp.
tcgggagtagtgccccaactggggtaacctttgggctccccgggcgcgtactccacctcaccc

SEQ ID NO: 9 - DNA, 63 bp, Streptomyces sp.
gggtgaggtggagtacgcgcccggggagcccaaaggttaccccagttggggcactactcccga

SEQ ID NO: 10 - DNA, 33 bp, Xenopus tropicalis
ggtgccagggcgtgcccttgggctccccgggcg

SEQ ID NO: 11 - DNA, 18 bp, Xenopus tropicalis
tgccagggcgtgcccttg

SEQ ID NO: 12 - DNA, 16 bp, Xenopus tropicalis
tgcccttgggctcccc

SEQ ID NO: 13 - DNA, 19 bp, Xenopus tropicalis
tgcccttgggctccccggg

SEQ ID NO: 14 - DNA, 16 bp, Xenopus tropicalis
cttgggctccccgggc

SEQ ID NO: 15 - DNA, 34 bp, Xenopus tropicalis
ccccaactggggtaacctttgagttctctcagtt

SEQ ID NO: 16 - DNA, 16 bp, Xenopus tropicalis
ctggggtaacctttga

SEQ ID NO: 17 - DNA, 16 bp, Xenopus tropicalis
tttgagttctctcagt

SEQ ID NO: 18 - DNA, 16 bp, Xenopus tropicalis
aacctttgagttctct

SEQ ID NO: 19 - DNA, 16 bp, Xenopus tropicalis
ctggggtaacctttga

SEQ ID NO: 20 - DNA, 24 bp, Xenopus tropicalis
aacctttgagttctctcagttctc

(19) United States
(12) Patent Application Publication
(10) Pub. No.: US 2007/0031390 A1
(43) Pub. Date: Feb. 8, 2007

(54) Methods and compositions for site-specific genomic expression of nucleic acid sequences

(76) Inventor: Daniel Weeks, Iowa City, IA (US)

Correspondence Address: VIKSNINS HARRIS & PADYS PLLP, P.O. BOX 111098, ST. PAUL, MN, 55111-1098, US

(21) Appl. No.: 11/436,357

(22) Filed: May 18, 2006

Related U.S. Application Data
(60) Provisional application No. 60/682,453, filed on May 18, 2005.

(51) Int. Cl. A61K 48/00; C12N 5/08; C12N 15/86

(52) U.S. Cl. 424/93.21; 435/456; 435/366

(57) ABSTRACT

The present invention is directed to methods and compositions for site-specific genomic expression of nucleic acid sequences.

### CROSS-REFERENCE TO RELATED APPLICATION

This application is related to and claims priority under 35 U.S.C. §119(e) to U.S. Provisional Application No. 60/682,453 filed on May 18, 2005, which is incorporated by reference herein.

### STATEMENT REGARDING FEDERALLY SPONSORED RESEARCH OR DEVELOPMENT

Work relating to this application was supported by a grant from the National Institutes of Health, RO1 GM 069944. The government may have certain rights in the invention.

### BACKGROUND OF THE INVENTION

The chromosomal DNA of eukaryotic organisms is thought to be organized into a series of higher-order regions or “domains” that define discrete units of compaction of chromatin, which is the complex of nucleoproteins interacting with eukaryotic nuclear DNA. In addition to providing a means for condensing the very large chromosomes of higher eukaryotes into a small nuclear volume, the domain organization of eukaryotic chromatin may have important consequences for gene regulation. The regulation of tissue-specific gene expression at the DNA level is mediated through an interaction between regulatory sequences in the DNA of eukaryotic cells and a complex of transcriptional factors (i.e., nucleoproteins) that are specific for a particular tissue type and for a particular gene. Further, the higher-order chromatin structure of tissue-specific genes is also regulated in a tissue-specific manner.

Higher-order chromatin domains may also define independent units of gene activity and regulation. For example, a discrete domain of eukaryotic chromatin is sometimes more than 100 kilobases in length and may encompass a particular gene or gene cluster. In those tissues where a given gene or gene cluster is active, the domain is sensitive to DNase I, thus lending support to the notion that the chromatin of an active domain is in a loose, decondensed configuration that is easily accessible to trans-acting factors. By contrast, in those tissues where the same gene is not active, the chromatin of the domain is in a tight configuration that is inaccessible to transacting factors. Thus, decondensing the higher order chromatin structure of a domain is required before regulatory factors can interact with target sequences, thereby determining the transcriptional competence of that domain.

It is desirable in basic and applied biology to perform efficient, site-specific integration of incoming DNA into the chromosomes of higher organisms. Recently strategies for chromosomal integration that take advantage of the high efficiency and tight sequence specificity of recombinase enzymes isolated from microorganisms have been described. In particular, a class of phage integrases that includes the phiC31 integrase (Kuhstoss et al., J. Mol. Biol. 222, 897-908 (1991); Rausch et al., Nucleic Acids Research 19, 5187-5189 (1991)) has been shown to function in mammalian cells (Groth et al., Proc. Natl. Acad. Sci. USA 97, 5995-6000 (2000)).

Such site-specific recombinase enzymes have long DNA recognition sites that are statistically unlikely to be present even in the large genomes of mammalian cells. However, it has been recently demonstrated that recombinase pseudo sites, i.e., sites with a significant degree of identity to the wild-type binding site for the recombinase, are present and can function in these genomes (Thyagarajan et al., Gene 244, 47-54 (2000)).

### SUMMARY OF THE INVENTION

One embodiment of the present invention is directed to compositions, and methods of using those compositions, to site-specifically integrate and express a polynucleotide sequence of interest in a genome of a eukaryotic cell.

The present invention provides a vector for site-specific integration of a polynucleotide sequence into an isolated eukaryotic cell's genome. The vector contains several elements including (a) one or more isolated polynucleotide encoding a chromatin insulator element, wherein the insulator element when flanking a gene to be inserted into a host chromosome insulates the transcriptional expression of the gene from one or more cis-acting regulatory sequences in chromatin into which the gene has been inserted; (b) a polynucleotide of interest operably linked to a eukaryotic promoter, and (c) a single recombination site, wherein the single recombination site comprises a polynucleotide sequence that recombines with a second recombination site in the genome of the isolated eukaryotic cell and the recombination occurs in the presence of a site-specific recombinase. Exemplary recombinases include phiC31 phage recombinase, TP901-1 phage recombinase, or R4 phage recombinase.

In one embodiment of the claimed vector, the insulator consists of a eukaryotic DNase I-hypersensitive site from the 5′ region of the chicken beta-globin gene locus. For example, the insulator element may be isolated from a 1.2 kilobase SacI-Sspl DNA.

In one embodiment, the insulator element used in the vector is isolated from a higher eukaryotic organism, such as a human. In certain embodiments, the promoter operably linked to the polynucleotide of interest is a tissue-specific promoter.

The present invention also provides a method of site-specifically integrating a nucleic acid into a genome of a cell of a multicellular organism. The method involves introducing the vector described above and a recombinase and/or a nucleic acid encoding a recombinase into a cell, and maintaining the cell under conditions sufficient for the recombination site to integrate into a genome attachment site in the genome of the cell by a recombination event mediated by the recombinase.

In certain embodiments, the genome attachment site is a pre-selected site in the genome. The cell used in the method may be a mammalian cell, such as a human cell. In certain embodiments, the promoter operably linked to the polynucleotide of interest is a tissue-specific promoter, resulting in tissue-specific expression of the target sequence. As used herein, the term “tissue-specific” means that the gene product of the target sequence is expressed in the target tissue, but is largely not expressed in other tissues. For example, the gene product is present at a level 10% greater than other tissues, 20%, 30%, 40%, 50%, 60%, 70%, 80%, 90%, 95%, 100%, or more than the non-targeted tissue.

The present invention further provides a kit for use in integrating a nucleic acid into a genome of a cell of a multicellular organism. The kit contains (a) a vector as described above; and (b) a recombinase or nucleic acid encoding a recombinase. The kit may further contain instructions for using the vector and the recombinase or nucleic acid encoding a recombinase in a method of modifying a genome of a cell of a multicellular organism.

The method comprises introducing (i) a circular targeting construct, comprising a first recombination site and the polynucleotide sequence of interest and at least one chromatin insulator element, and (ii) a site-specific recombinase into the eukaryotic cell, wherein the genome of the cell comprises a second recombination site native to the genome and recombination between the first and second recombination sites is facilitated by the site-specific recombinase, wherein the chromatin insulator element prevents chromosomal position effect. This allows expression of a target polynucleotide contained in the circular targeting construct. The cell is maintained under conditions that allow recombination between the first and second recombination sites and the recombination is mediated by the site-specific recombinase. The result of the recombination is site-specific integration and expression of the polynucleotide sequence of interest in the genome of the eukaryotic cell.

The recombinase may be introduced into the cell before, concurrently with, or after introducing the circular targeting construct. Further, the circular targeting construct may comprise other useful components, such as a bacterial origin of replication and/or a selectable marker.

In certain embodiments, the site-specific recombinase is a recombinase encoded by a phage selected from the group consisting of phiC31, TP901-1, and R4. The recombinase may facilitate recombination between a sequence identical to or similar enough to function like (i. e., a pseudo attB site) the bacterial genomic recombination site (attB) and a sequence identical to or similar enough to function like (i.e., a pseudo attP site) the phage genomic recombination site (attP), and (i) the second recombination site may comprise an attP site or a pseudo-attP site, and (ii) the first recombination site may comprise the attB or pseudo attB site or (i) the second recombination site may comprise an attB or pseudo-attB site, and (ii) the first recombination site may comprise the attP or pseudo attP site.

In yet a further embodiment, the site-specific recombinase is introduced into the cell as a polypeptide. In alternative embodiments, the site-specific recombinase in introduced into the cell as a polynucleotide encoding the recombinase and an expression cassette, optionally carried on a transient expression vector, comprises the polynucleotide encoding the recombinase.

In another embodiment, the invention is directed to a vector for site-specific integration of a polynucleotide sequence into the genome of a eukaryotic cell. The vector comprises (i) a circular backbone vector, (ii) a polynucleotide of interest operably linked to a eukaryotic promoter, (iii) a first recombination site, wherein the genome of the cell comprises a second recombination site native to the genome and recombination between the first and second recombination sites is facilitated by a site-specific recombinase, and (iv) an isolated polynucleotide encoding a chromatin insulator element, wherein the insulator element when flanking a gene to be inserted into a host chromosome insulates the transcriptional expression of the gene from one or more cis-acting regulatory sequences in chromatin into which the gene has been inserted.

In certain embodiments, the recombinase normally facilitates recombination between a bacterial genomic recombination site (attB) and a phage genomic recombination site (attP) and the first recombination site may be either attB or attP.

In still another embodiment, the invention is directed to a kit for site-specific integration of a polynucleotide sequence into the genome of a eukaryotic cell. The kit comprises (i) a vector as described above and (ii) a site-specific recombinase.

In another embodiment, the invention is directed to a eukaryotic cell having a modified genome. The modified genome contains an isolated polynucleotide encoding a chromatin insulator element and an integrated polynucleotide sequence of interest whose integration was mediated by a recombinase, wherein the integration was into a recombination site native to the eukaryotic cell genome and the integration created a recombination-product site comprising the polynucleotide sequence.

In further embodiments, the subject invention is directed to transgenic plants and animals comprising at least one cell as described above, as well as methods of producing the same.

In yet other embodiments, the invention is directed to methods of treating a disorder in a subject in need of such treatment. The method comprises site-specifically integrating a polynucleotide sequence of interest flanked by chromatin insulator elements in a genome of at least one cell of the subject, wherein the polynucleotide facilitates production of a product that treats the disorder in the subject. The site-specific integration may be carried out in vivo in the subject, or ex vivo in cells and the cells are then introduced into the subject.

A further embodiment of the invention comprises cells, tissues, transgenic animals and/or plants whose genomes have been modified using the methods described herein.

In another aspect, the present invention provides a method of modifying a genome of a cell. In the method, an attB or an attP recombination site is inserted into the genome of a cell, wherein (i) the recombination site is recognized by a recombinase, and (ii) the cell normally does not comprise or contain a well functioning attB or attP site. The vectors described herein and above are useful in the practice of this aspect of the invention. In a preferred embodiment, the cell that is being modified is a eukaryotic cell.

In yet another aspect, the present invention provides expression cassettes, comprising a polynucleotide encoding a site-specific recombinase, wherein (i) the recombinase is encoded by a phage (typically selected from the group consisting of .phi.C31, TP901-1, and R4) and the recombinase is operably linked to a eukaryotic promoter. The vectors described herein and above are useful in the practice of this aspect of the invention.

These and other embodiments of the present invention will readily occur to those of ordinary skill in the art in view of the disclosure herein.

Throughout this application, various publications, patents, and published patent applications are referred to by an identifying citation. The disclosures of these publications, patents, and published patent specifications referenced in this application are hereby incorporated by reference into the present disclosure to more fully describe the state of the art to which this invention pertains.

The practice of the present invention will employ, unless otherwise indicated, conventional techniques of molecular biology, microbiology, cell biology and recombinant DNA, which are within the skill of the art. See, e.g., Sambrook, Fritsch, and Maniatis, Molecular Cloning: A Laboratory Manual, 2nd edition (1989); Current Protocols In Molecular Biology, (F. M. Ausubel et al. eds., 1987); the series Methods In Enzymology (Academic Press, Inc.); PCR 2: A Practical Approach (M. J. McPherson, B. D. Hames and G. R. Taylor eds., 1995) and Animal Cell Culture (R. I. Freshney. Ed., 1987).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise. Thus, for example, reference to “an enzyme” includes a mixture of two or more such agents.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1 is a cartoon showing the phiC31 integrase reaction.

FIG. 2 provides phiC31 integrase att sites.

FIG. 3 provides phiC31 integrase att sites in Xenopus tropicalis.

FIG. 4 provides a schematic drawing of the method of using phiC31 integrase to make transgenic tadpoles.

FIG. 5 provides phiC31 integrase mediated integration. Typical expression patterns were seen in Stage 46 embryos (about 7 days post-fertilization).

FIG. 6 is a cartoon showing that enhancer elements can activate gene relatively independently of position or orientation.

FIG. 7 shows insulator control position effects.

FIG. 8 shows that uniform expression of GFP was achieved when the transgene was flanked with insulators.

FIG. 9 shows the results of when the inventors assayed the expression of CMV-GFP reporter plasmid in tadpoles, where the transcription unit is bounded by insulator elements. In each panel is a brightfield and a fluorescent photograph of embryos treated at the single-cell stage and assayed at Stage 46. (A) Non-Injected, (B) injected with insulated CMV-GFP plasmid alone, and (C) injected with insulated, CMV-GFP and integrase mRNA.

FIG. 10 shows that when the CMV promoter was replaced with a minimal Xenopus crystallin lens promoter in the insulated GFP reporter construct, the construct induced lens-specific GFP expression.

FIG. 11 provides GFP expression data for injected Xenopus stage 46 glowing embryos.

### DETAILED DESCRIPTION OF THE INVENTION

The invention encompasses isolated or substantially purified nucleic acid or protein compositions. In the context of the present invention, an “isolated” or “purified” DNA molecule or RNA molecule or an “isolated” or “purified” polypeptide is a DNA molecule, RNA molecule, or polypeptide that exists apart from its native environment and is therefore not a product of nature. An isolated DNA molecule, RNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, or 5% (by dry weight) of contaminating protein. When the protein of the invention, or biologically active portion thereof, is recombinantly produced, culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

“Naturally occurring,” “native” or “wildtype” are used to describe an object that can be found in nature as distinct from being artificially produced. For example, a protein or nucleotide sequence present in an organism (including a virus), which can be isolated from a source in nature and which has not been intentionally modified by a person in the laboratory, is naturally occurring.

The term “chimeric” refers to a gene or DNA that contains 1) DNA sequences, including regulatory and coding sequences that are not found together in nature or 2) sequences encoding parts of proteins not naturally adjoined, or 3) parts of promoters that are not naturally adjoined. Accordingly, a chimeric gene may include regulatory sequences and coding sequences that are derived from different sources, or include regulatory sequences and coding sequences derived from the same source, but arranged in a manner different from that found in nature.

A “transgene” refers to a gene that has been introduced into the genome by transformation. Transgenes include, for example, DNA that is either heterologous or homologous to the DNA of a particular cell to be transformed. Additionally, transgenes may include native genes inserted into a non-native organism, or chimeric genes.

The term “endogenous gene” refers to a native gene in its natural location in the genome of an organism.

A “foreign” gene refers to a gene not normally found in the host organism that has been introduced by gene transfer.

The terms “protein,” “peptide” and “polypeptide” are used interchangeably herein.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest but may also code for a functional RNA of interest, for example an antisense RNA, a nontranslated RNA in the sense or antisense direction, or an siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of an regulatable promoter that initiates transcription only when the host cell is exposed to some particular stimulus. In the case of a multicellular organism, the promoter can also be specific to a particular tissue or organ or stage of development.

Such expression cassettes can include a transcriptional initiation region linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, regulatable promoters and viral promoters. Examples of promoters that may be used in the present invention include CMV, RSV, pol II and pol III promoters.

“Promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

The terms “cis-acting sequence” and “cis-acting element” refer to DNA or RNA sequences whose functions require them to be on the same molecule. An example of a cis-acting sequence on the replicon is the viral replication origin.

The terms “trans-acting sequence” and “trans-acting element” refer to DNA or RNA sequences whose function does not require them to be on the same molecule.

“Chromosomally-integrated” refers to the integration of a foreign gene or nucleic acid construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm. Non-limiting examples of such mathematical algorithms are the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, JMB, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)), are based on the algorithm of Karlin and Altschul supra.

Software for performing BLAST analyses is publicly available through the National Center for Biotechnology Information on the world-wide-web at ncbi.nlm.nih.gov. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized as described in Altschul et al., Nuc. Acids Res. 25:3389-3402 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world-wide-web at www.ncbi.nlm.nih.gov. Alignment may also be performed manually by inspection.

For purposes of the present invention, comparison of nucleotide sequences for determination of percent sequence identity to the promoter sequences disclosed herein is made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. Optimal alignment may be conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl (Anal Biochem. 138(2):267-84 (1984)); Tₘ 81.5° C.+16.6 (log M) +0.41 (% GC)−0.61 (% form) −500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), the SSC concentration may be increased so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, N.Y. (1993). Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook and Russell, Molecular Cloning: A Laboratory Manual, Cold Spring Harbor Laboratory Press Cold Spring Harbor, N.Y. (2001), for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

The term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells, and organisms comprising transgenic cells are referred to as “transgenic organisms”.

“Transformed”, “transduced”, “transgenic”, and “recombinant” refer to a host cell or organism into which a heterologous nucleic acid molecule has been introduced. The nucleic acid molecule can be stably integrated into the genome generally known in the art and are disclosed in Sambrook and Russell, infra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

“Treating” as used herein refers to ameliorating at least one symptom of, curing and/or preventing the development of a disease or a condition.

I. Chromatin Insulator Elements

The present invention uses chromatin insulator elements, such as the specific 5′ constitutive hypersensitive site of the chicken beta-globin domain (U.S. Pat. No. 5,610,053, incorporated by reference herein). These chromatin insulator elements are a pure chromatin insulator elements that do not influence gene expression in a positive or negative manner. The insulator element prevents or blocks the spread of the LCRs' disruption of chromatin. The insulator element is a control element that insulates the transcription of genes placed within its range of action. In one embodiment, the insulator element is a DNA segment that encompasses a 1.2 kb fragment of DNA isolated from the far 5′ end of the chicken beta-globin locus and includes the chicken 5′ constitutive hypersensitive site (5′HS4). The insulator element contains a “core” DNA sequence of about 242 bp to 250 bp, which also has demonstrable pure insulator activity. The 5′HS4 site is located about 12 to about 15 kb 5′ of the rho-globin gene and about 18 to about 20 kb 5′ of the chicken beta-globin gene. An example of a suitable 242 pb insulator element is the following:

(SEQ ID No: 1)

AGGGACAGCCCCCCCCCAAAGCCCCCAGGGATGTAATTACGTCCCTCCCC
CGCTAGGGCAGCAGCGAGCCGCCCGGGGCTCCGGTCCGGTCCGGCGCTCC
CCCGCATCCCCGAGCCGGCAGCGTGCGGGGACAGCCCGGGCACGGGGAAG
GTGGCACGGGATCGCTTTCCTCTGAACGCTTCTCGCTGCTCTTTGAGCCT
GCAGACACCTGGGGGGATACGGGGAAAAAAGCTTTAGGCTGA,.

In its natural position, the chromatin insulator element presumably buffers the genes and the regulatory machinery of one domain from the cis-acting influence(s) of the chromatin structure and the regulatory machinery of an adjacent domain. In genetic constructs of the invention, the insulator element can exert its optimal insulation or buffering effects on a reporter gene when the element, or a DNA fragment containing the element (i.e., the 1.2 kb DNA fragment), is inserted on either side of a reporter gene, such that the insulator is positioned at least about 200 bp to about 1 kb, for example about 330 bp, from the promoter and at least about 1 kb to about 5 kb, preferably about 2.7 kb, from the promoter, at the 3′ end of the reporter gene. In addition, more than one insulator element may be positioned in tandem on either side of a reporter gene. Those skilled in the art will be aware that the distances of the insulator element from the promoter and the reporter gene in the constructs are provided for guidance and may depend upon the relative sizes of the reporter gene or genes, the promoter, and the enhancer, or LCR, used in the constructs.

The insulator elements can be employed to provide novel constructs for efficient isolation and protection of genes and for the production of a particular protein or other molecule encoded by a gene used in the constructs in cells. The insulator element of the invention may also be used to insulate particular genes introduced and subsequently expressed in transgenic animals, such as fruit flies (e.g., Drosophila melanogaster), mice, rodents, and the like. Constructs containing the insulator elements of the invention may be introduced into early fetal or embryonic cells for the production of transgenic animals containing the functional insulator element and reporter gene transcription unit, as described further hereinbelow. By insulating a gene or genes introduced into the transgenic animal, the expression of the gene(s) will be protected from negative or inappropriately positive regulatory influences in the chromatin at or near the site of integration.

In general, the constructs of the present invention contain a higher eukaryotic insulator element, an enhancer element or LCR, and a transcription unit comprising, at a minimum, a gene of interest, for example, a gene encoding a protein or precursor thereof, and a promoter to drive the transcription of the gene of interest, and other sequences necessary or required for proper gene transcription and regulation (e.g., start and stop sites, splice sites, polyadenylation signal, and an origin of replication). The enhancer element or LCR is located in sufficient proximity to the transcription unit to enhance the transcription thereof. The constructs may contain more than one insulator element, preferably in tandem, which are positioned so as to insulate the reporter gene and its transcription unit from surrounding DNA at the site of integration.

Transcriptionally competent transcription units can be made by conventional techniques. In general, the insulator element is placed in sufficient proximity to the enhancer or LCR so that it is functionally active to buffer the effects of a cis-acting DNA region on the promoter of the transcription unit. In some cases, the insulator can be placed distantly from the transcription unit. In addition, the optimal location of the insulator element can be determined by routine experimentation for any particular DNA construct. The function of the insulator element is substantially independent of its orientation, and thus the insulator can function when placed in genomic or reverse genomic orientation with respect to the transcription unit, as long as the insulator is placed preferably on both sides of a gene so as to insulate the gene from the effects of cis-acting DNA sequences of chromatin.

In another embodiment, the constructs may contain more than one reporter gene whose expression is to be insulated by the insulator elements. In the case where two genes are to be transcribed and expressed at different levels, the construct may contain different enhancers to regulate the transcription of each gene. Accordingly, one enhancer could be a weak enhancer and the other enhancer could be a strong enhancer to allow the differential expression of the two genes in the same construct following integration into the DNA. Alternatively, the promoter of one gene can be inducible, while the promoter of a second gene can be non-inducible, or the second promoter can also be inducible, but can be induced by a different agent.

Those skilled in the art will appreciate that a variety of enhancers, promoters, and genes are suitable for use in the constructs of the invention, and that the constructs will contain the necessary start, termination, and control sequences for proper transcription and processing of the gene of interest when the construct is introduced into a mammalian or a higher eukaryotic cell. The constructs may be introduced into cells by a variety of gene transfer methods known to those skilled in the art, for example, gene transfection, microinjection, electroporation, and infection. In addition, it is envisioned that the invention can encompass all or a portion of a viral sequence-containing vector, such as those described in U.S. Pat. No. 5,112,767, for targeted delivery of genes to specific tissues. The constructs of the invention may integrate stably into the genome of specific and targeted cell types.

Further, the DNA construct comprising the insulator element, enhancer or LCR, and transcription unit may be inserted into or assembled within a vector such as a plasmid or virus, as mentioned above. The construct can be assembled or spliced into any suitable vector or cosmid for incorporation into the host cell of interest. The vectors may contain a bacterial origin of replication so that they can be amplified in a bacterial host. The vectors may also contain, in addition to a selectable marker for selection of transfected cells, as in the exemplary constructs, another expressible and selectable gene of interest.

Vectors can be constructed which have the insulator element in appropriate relation to an insertion region for receiving DNA encoding a protein or precursor thereof. The insertion region can contain at least one restriction enzyme recognition site.

Other chromatin insulator elements (e.g. both tissue-specific and non-specific) may be used in the constructs of the present invention, either by cloning and isolating eukaryotic constitutive hypersensitive sites having sequences similar to the chicken and human insulator elements disclosed herein, or by using other sequences known or tested to be constitutive hypersensitive sites that function as insulator elements.

II. Recombinase Enzymes and Recognition Sites

“Recombinase” as used herein refers to a group of enzymes that can facilitate site specific recombination between defined sites, where the sites are physically separated on a single DNA molecule or where the sites reside on separate DNA molecules. The DNA sequences of the defined recombination sites are not necessarily identical. Within this group are several subfamilies including “Integrase” (including, for example, Cre and lambda integrase) and “Resolvase/Invertase” (including, for example, phiC31 integrase, R4 integrase, and TP-901 integrase). A detailed description of various recombinases is provided in U.S. Pat. No. 6,632,672 and U.S. Pat. No. 6,808,925 (incorporated by reference herein).

By “pseudo-recombination site (RS/P)” is meant a site at which recombinase can facilitate recombination even though the site may not have a sequence identical to the sequence of its wild-type recombination site. A pseudo-recombination site is typically found in an organism heterologous to the native phage/bacterial system. For example, a phiC31 integrase and vector carrying a phiC31 wild-type recombination site can be placed into a eukaryotic cell. The wild-type recombination sequence aligns itself with a sequence in the eukaryotic cell genome and the integrase facilitates a recombination event. When the sequence from the genomic site, in the eukaryotic cell, where the integration of the vector took place (via a recombination event between the wild-type recombination site in the vector and the genome) is examined, the sequence at the genomic site typically has some identity to but may not be identical with the wild-type bacterial genome recombination site. The recombination site in the eukaryotic cell is considered to be a pseudo-recombination site at least because the eukaryotic cell is heterologous to the normal phage/bacterial cell system. The size of the pseudo-recombination site can be determined through the use of a variety of methods including, but not limited to, (i) sequence alignment comparisons, (ii) secondary structural comparisons, (iii) deletion or point mutation analysis to find the functional limits of the pseudo-recombination site, and (iv) combinations of the foregoing. Pseudo-recombination sites typically occur naturally in the genomes of eukaryotic cells (i.e., the sites are native to the genome) and are functionally identified as described herein. A detailed description of various recombination sites recognized by recombinases is provided in U.S. Pat. No. 6,632,672 and U.S. Pat. No. 6,808,925 (incorporated by reference herein).

III. Nucleic Acid Molecules of the Invention

Sources of nucleotide sequences from which the present nucleic acid molecules can be obtained include any vertebrate, such as mammalian, cellular source.

As discussed above, the terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule encoding a target molecule that is transcribed into an mRNA. Thus, the RNA or DNA is “isolated” in that it is free from at least one contaminating nucleic acid with which it is normally associated in the natural source of the RNA or DNA and is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid”, e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from a source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. See Lawn et al., Nucleic Acids Res., 9, 6103 (1981), and Goeddel et al., Nucleic Acids Res., 8, 4057 (1980). Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

The DNA template can be generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153, 3 (1987). Thus, the DNA that is to be mutated may be inserted into one of these vectors to generate single-stranded template. Production of the single-stranded template is described in Chapter 3 of Sambrook and Russell, 2001. Alternatively, single-stranded DNA template may be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the DNA, and the other strand (the original template) encodes the native, unaltered sequence of the DNA. This heteroduplex molecule is then transformed into a suitable host cell, usually a prokaryote such as E. coli JM101. After the cells are grown, they are plated onto agarose plates and screened using the oligonucleotide primer radiolabeled with 32-phosphate to identify the bacterial colonies that contain the mutated DNA. The mutated region is then removed and placed in an appropriate vector, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(*S) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(*S) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. Coli JM101.

IV. Expression Cassettes of the Invention

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the DNA, or the like. Such elements may be included in the DNA as desired to obtain the optimal performance of the DNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

Operably linked nucleic acids are nucleic acids placed in a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

In order to prevent any packaging of AAV genomic sequences containing the rep and cap genes, a plasmid containing the rep and cap DNA fragment can be modified by the inclusion of a stuffer fragment into the AAV genome which causes the DNA to exceed the length for optimal packaging. Thus, in certain embodiments, the helper fragment is not packaged into AAV virions. This is a safety feature, ensuring that only a recombinant AAV vector genome that does not exceed optimal packaging size is packaged into virions. An AAV helper fragment that incorporates a stuffer sequence can exceed the wild-type genome length of 4.6 kb, and lengths above 105% of the wild-type will generally not be packaged. The stuffer fragment can be derived from, for example, such non-viral sources as the Lac-Z or beta-galactosidase gene.

The general methods for constructing recombinant DNA that can transfect target cells are well known to those skilled in the art, and the same compositions and methods of construction may be utilized to produce the DNA useful herein. For example, Sambrook and Russell, infra, provides suitable methods of construction.

The recombinant DNA can be readily introduced into the host cells, e.g., mammalian, bacterial, yeast or insect cells by transfection with an expression vector composed of DNA encoding the target gene by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. The DNA is introduced into host cells via a vector. The host cell is may be of eukaryotic origin, e.g., plant, mammalian, insect, yeast or fingal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described hereinbelow, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like. See, for example, U.S. Pat. Nos. 5,350,674 and 5,585,362.

As discussed above, a “transfected”, “or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the preselected DNA segment is being expressed. Expression may be evaluated by specifically identifying the peptide products of the introduced recombinant DNA sequences or evaluating the phenotypic changes brought about by the expression of the introduced recombinant DNA segment in the host cell.

The instant invention provides a cell expression system for expressing exogenous nucleic acid material in a mammalian recipient. The expression system, also referred to as a “genetically modified cell”, comprises a cell and an expression vector for expressing the exogenous nucleic acid material. The genetically modified cells are suitable for administration to a mammalian recipient, where they replace the endogenous cells of the recipient. Thus, the genetically modified cells are non-immortalized and are non-tumorigenic.

According to one embodiment, the cells are transfected or otherwise genetically modified ex vivo. The cells are isolated from a mammal (such as a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

As used herein, “exogenous nucleic acid material” refers to a nucleic acid or an oligonucleotide, either natural or synthetic, which is not naturally found in the cells; or if it is naturally found in the cells, is modified from its original or native form. Thus, “exogenous nucleic acid material” includes, for example, a non-naturally occurring nucleic acid that can be transcribed into an anti-sense RNA, a siRNA, as well as a “heterologous gene” (i.e., a gene encoding a protein that is not expressed or is expressed at biologically insignificant levels in a naturally-occurring cell of the same type). To illustrate, a synthetic or natural gene encoding human erythropoietin (EPO) would be considered “exogenous nucleic acid material” with respect to human peritoneal mesothelial cells since the latter cells do not naturally express EPO. Still another example of “exogenous nucleic acid material” is the introduction of only part of a gene to create a recombinant gene, such as combining an regulatable promoter with an endogenous coding sequence via homologous recombination.

V. Promoters of the Invention

As described herein, an expression cassette of the invention contains, inter alia, a promoter. Such promoters include the CMV promoter, as well as the RSV promoter, SV40 late promoter and retroviral LTRs (long terminal repeat elements), or tissue specific promoters. Many other promoter elements well known to the art, such as regulatable promoters may be employed in the practice of the invention.

VI. Methods for Introducing the Expression Cassettes of the Invention into Cells

The condition amenable to gene inhibition therapy may be a prophylactic process, i.e., a process for preventing disease or an undesired medical condition. Thus, the instant invention embraces a system for delivering a target DNA that has a prophylactic function (i.e., a prophylactic agent) to the mammalian recipient.

The inhibitory nucleic acid material (e.g., an expression cassette encoding a target DNA directed to a gene of interest) can be introduced into the cell ex vivo or in vivo by genetic transfer methods, such as transfection or transduction, to provide a genetically modified cell. Various expression vectors (i.e., vehicles for facilitating delivery of exogenous nucleic acid into a target cell) are known to one of ordinary skill in the art.

As used herein, “transfection of cells” refers to the acquisition by a cell of new nucleic acid material by incorporation of added DNA. Thus, transfection refers to the insertion of nucleic acid into a cell using physical or chemical methods. Several transfection techniques are known to those of ordinary skill in the art including: calcium phosphate DNA co-precipitation (Methods in Molecular Biology, 7, Gene Transfer and Expression Protocols, Ed. E. J. Murray, Humana Press (1991)); DEAE-dextran Methods in Molecular Biology (Methods in Molecular Biology, supra); electroporation (Methods in Molecular Biology, supra); cationic liposome-mediated transfection (Methods in Molecular Biology, supra); and tungsten particle-facilitated microparticle bombardment (Johnston, Nature, 346, 776 (1990)). Strontium phosphate DNA co-precipitation (Brash et al., Molec. Cell. Biol., 7, 2031 (1987)) is also a transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous nucleic acid material contained within the retrovirus is incorporated into the genome of the transduced cell. A cell that has been transduced with a chimeric DNA virus (e.g., an adenovirus carrying a cDNA encoding a therapeutic agent), will not have the exogenous nucleic acid material incorporated into its genome but will be capable of expressing the exogenous nucleic acid material that is retained extrachromosomally within the cell.

The exogenous nucleic acid material can include the nucleic acid encoding the target DNA together with a promoter to control transcription. The promoter characteristically has a specific nucleotide sequence necessary to initiate transcription. The exogenous nucleic acid material may further include additional sequences (i.e., enhancers) required to obtain the desired gene transcription activity. For the purpose of this discussion an “enhancer” is simply any non-translated DNA sequence that works with the coding sequence (in cis) to change the basal transcription level dictated by the promoter. The exogenous nucleic acid material may be introduced into the cell genome immediately downstream from the promoter so that the promoter and coding sequence are operatively linked so as to permit transcription of the coding sequence. An expression vector can include an exogenous promoter element to control transcription of the inserted exogenous gene. Such exogenous promoters include both constitutive and regulatable promoters.

Naturally-occurring constitutive promoters control the expression of essential cell functions. As a result, a nucleic acid sequence under the control of a constitutive promoter is expressed under all conditions of cell growth. Constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA, 88 4626 (1991)), adenosine deaminase, phosphoglycerol kinase (PGK), pyruvate kinase, phosphoglycerol mutase, the beta-actin promoter (Lai et al., Proc. Natl. Acad. Sci. USA, 86, 10006 (1989)), and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others.

Nucleic acid sequences that are under the control of regulatable promoters are expressed only or to a greater or lesser degree in the presence of an inducing or repressing agent, (e.g., transcription under control of the metallothionein promoter is greatly increased in presence of certain metal ions). Regulatable promoters include responsive elements (REs) that stimulate transcription when their inducing factors are bound. For example, there are REs for serum factors, steroid hormones, retinoic acid, cyclic AMP, and tetracycline and doxycycline. Promoters containing a particular RE can be chosen in order to obtain an regulatable response and in some cases, the RE itself may be attached to a different promoter, thereby conferring regulatability to the encoded nucleic acid sequence. Thus, by selecting the appropriate promoter (constitutive versus regulatable; strong versus weak), it is possible to control both the existence and level of expression of a nucleic acid sequence in the genetically modified cell. If the nucleic acid sequence is under the control of an regulatable promoter, delivery of the therapeutic agent in situ is triggered by exposing the genetically modified cell in situ to conditions for permitting transcription of the nucleic acid sequence, e.g., by intraperitoneal injection of specific inducers of the regulatable promoters which control transcription of the agent. For example, in situ expression of a nucleic acid sequence under the control of the metallothionein promoter in genetically modified cells is enhanced by contacting the genetically modified cells with a solution containing the appropriate (i.e., inducing) metal ions in situ.

In addition to at least one promoter and at least one heterologous nucleic acid sequence encoding the target sequence, the expression vector may include a selection gene, for example, a neomycin resistance gene, for facilitating selection of cells that have been transfected or transduced with the expression vector.

Cells can also be transfected with two or more expression vectors, at least one vector containing the nucleic acid sequence(s) encoding the target sequence, the other vector containing a selection gene. The selection of a suitable promoter, enhancer, selection gene and/or signal sequence is deemed to be within the scope of one of ordinary skill in the art without undue experimentation.

The following discussion is directed to various utilities of the instant invention. For example, the instant invention has utility as an expression system suitable for silencing the expression of gene(s) of interest.

The instant invention also provides various methods for making and using the above-described genetically-modified cells.

The instant invention also provides methods for genetically modifying cells of a mammalian recipient in vivo. According to one embodiment, the method comprises introducing an expression vector for expressing a target sequence in cells of the mammalian recipient in situ by, for example, injecting the vector into the recipient.

The invention will now be illustrated by the following non-limiting Example.

### EXAMPLE 1

### Utilization of a Phase Integrase to Make Transgenic Xenopus Embryos

There are many reasons to use the embryos of the frog Xenopus laevis for studies on early heart development. The timing of gene expression, conservation of particular gene products and the formation of a three chambered heart all provide opportunities to examine specific regulatory and morphological events. Although possible, the creation of transgenic animals has some practical drawbacks that has limited its use in Xenopus. As currently practiced, demembranated sperm nuclei are mixed with DNA of interest and injected into unfertilized eggs. The integration site and copy number are difficult to control while inserting DNA fragments into sperm nuclei and the maturation of the embryos into fertile adults is relatively slow. We describe here our studies using the phiC31 integrase to generate transgenic embryos, and suggest that the efficiency of this process provides an alternative method to make transgenic embryos for studies on heart development.

The bacteriophage phiC31 encodes an integrase that allows it to lysogenize into Streptomyces ambofaciens. More recently the phiC31integrase system has been used to integrate genes into mouse cells, human cells and Drosophila. The site of genomic integration is specific for a 35-42 nucleotide sequence referred to as the attP site. Utilizing this system, we have introduced a 5-kilobase segment of DNA containing a CMV driven GFP into the Xenopus laevis genome. Many of the green fluorescent embryos matured into tadpoles suggesting that the insertion does not interfere with normal development.

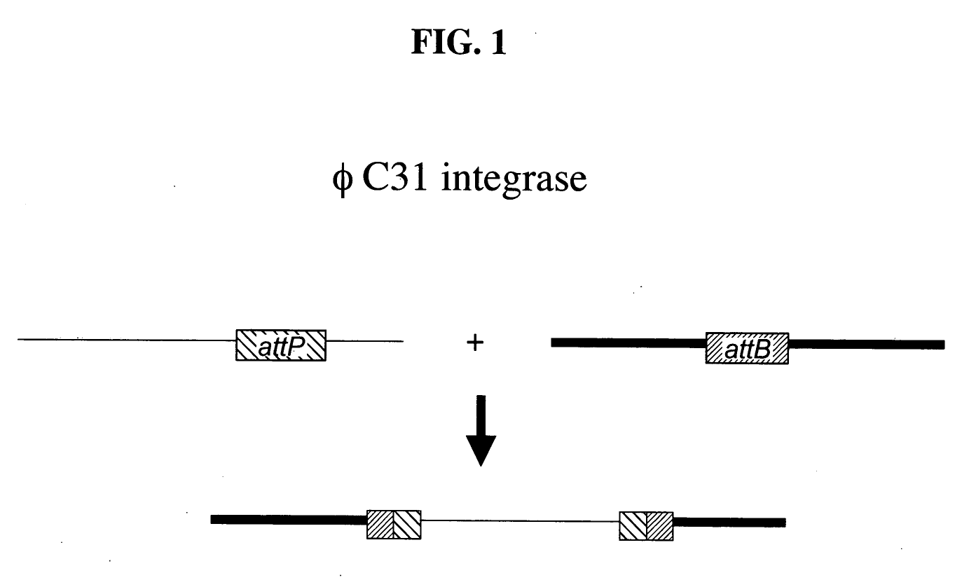

The phiC31 integrase comes from the temperate phage phiC31 (complete genome is 41491 bp, Accession: NC_{—}001978) that infects streptomyces. Insertion of the phage into the bacterial genome is mediated by integrase that utilizes two different DNA sequences, the attP site in the phage and the attB site in the bacteria. The integrase recognizes a sequence in the phage genome, phage attachment site, attp. It also recognizes a sequence in the bacterial genome, i.e., the bacterial attachment site attB. It catalyzes a recombination between an attB site and an attP site. This enzyme does not require any accessory proteins, and does not catalyze reverse reaction. FIG. 1 is a cartoon showing the phiC31 integrase reaction.

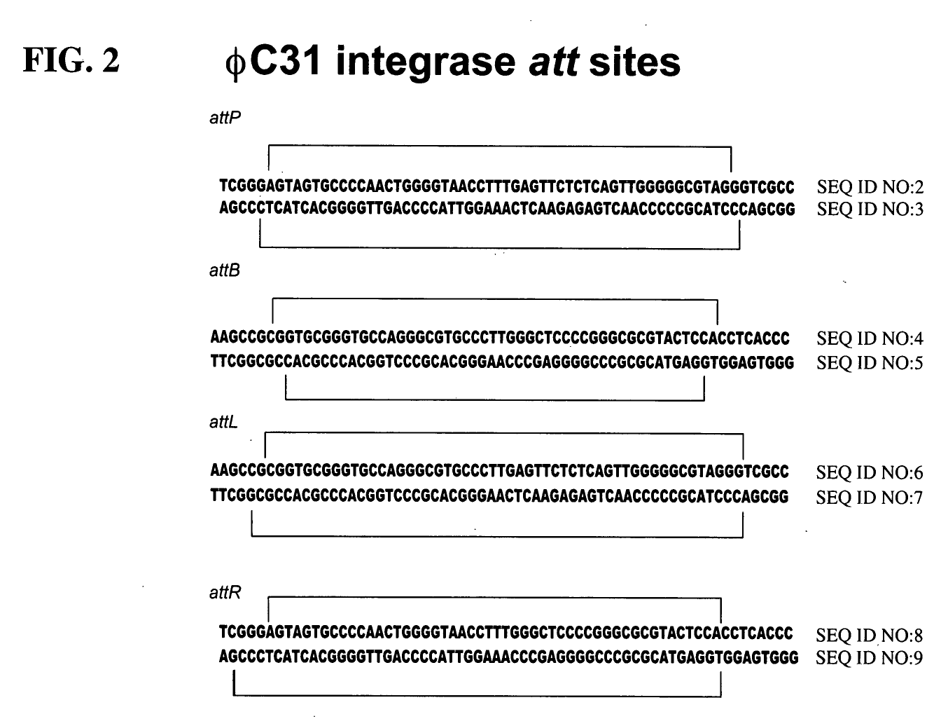

FIG. 2 provides phiC31 integrase att sites. The core TTG is the cross-over region. About 50 bp are contacted by the integrase. Integrase binds to all four sites depicted in FIG. 2 with the same affinity, but only attB/attP recombination is catalyzed. The minimal attB size is 34 bp, and the minimal attP size is 39 bp.

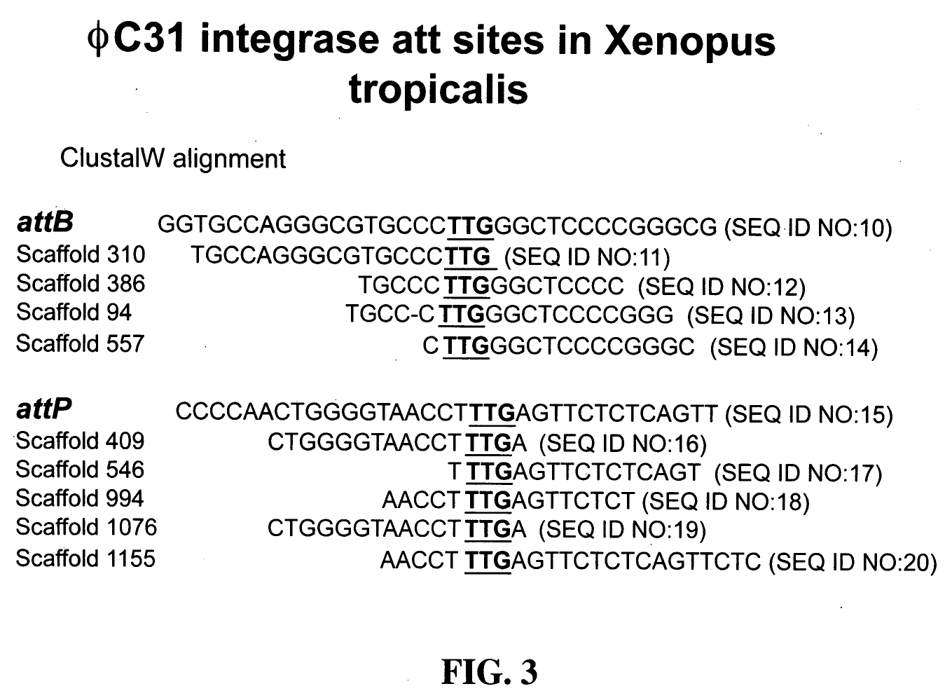

The enzyme phiC31 integrase is used for site-specific integration. PhiC31 integrase can be used for efficient integration into the chromosomes of human and mouse cells. Cells with inserted attP site have a frequency of integration of 10- to 20-fold above random integration. Also, the frequency of integration is 5- to 10-fold above random integration for endogenous sites. “Pseudo” attP sites have partial identity (56-24%) to attP sites. There are 57 identified pseudo attP sites in the mouse genome, and 31 identified pseudo attP sites in the human genome. In the preferential site, 14 out 25 bp are identical. There are about 10²-10³ pseudo attP sites in a mammalian genome. Integration is less efficient when attP containing plasmid was cotransfected into cells with attB site. FIG. 3 provides the known phiC31 integrase att sites in Xenopus tropicalis.

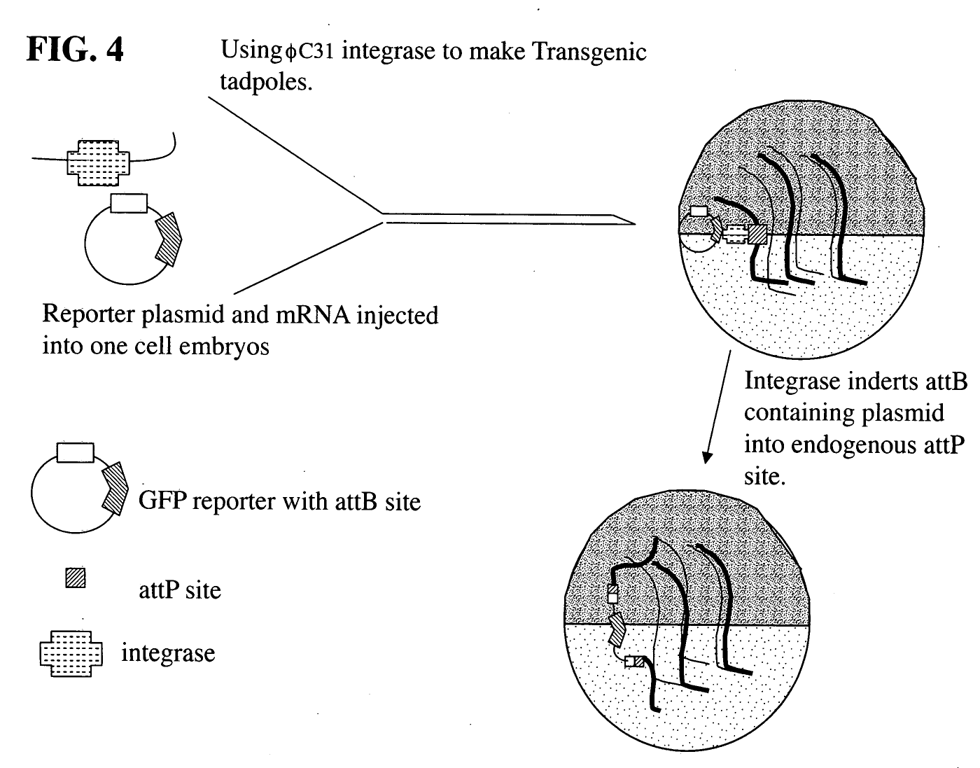

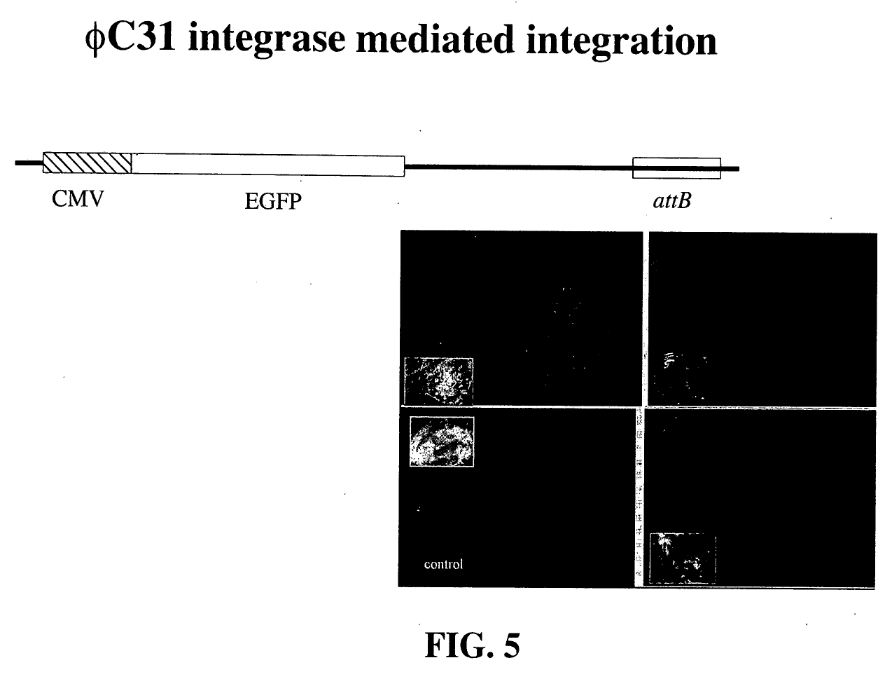

The present inventors used phiC31 integrase to make transgenic tadpoles. See FIG. 4. A reporter plasmid and mRNA is injected into single-cell embryos. Integrase inserts attB containing plasmid into endogenous attP site. Specifically, a CMV-GFP reporter plasmid containing an attB site was coinjected with phiC31 integrase mRNA into single-cell embryos, and then the tadpoles were assayed for GFP (FIG. 5). Uniform expression of GFP was not achieved, possibly because of chromosomal position effects.

DNA was extracted from embryos at stage 47, and cut with a restriction enzyme with a single site in the reporter plasmid. Linear plasmid yields a band 5050 bp, but integration will yield bands that do not correspond to 5050 bp. The Southern blot analysis showing the hybridization of reporter plasmid probe to extracted DNA revealed bands of different lengths. The data was consistent with integration of the reporter into the genome, and that there are at least three different integration sites.

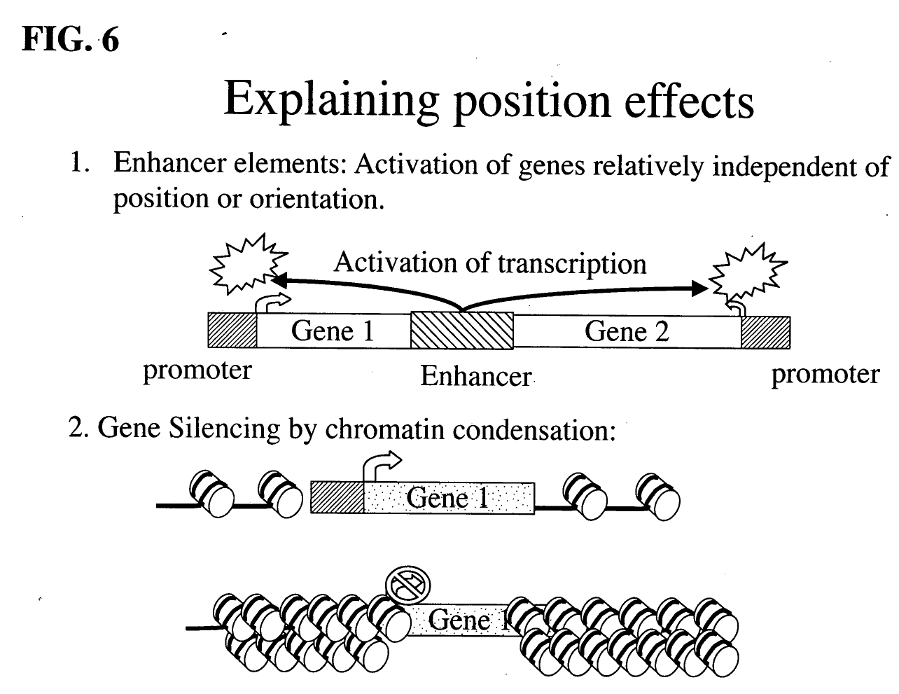

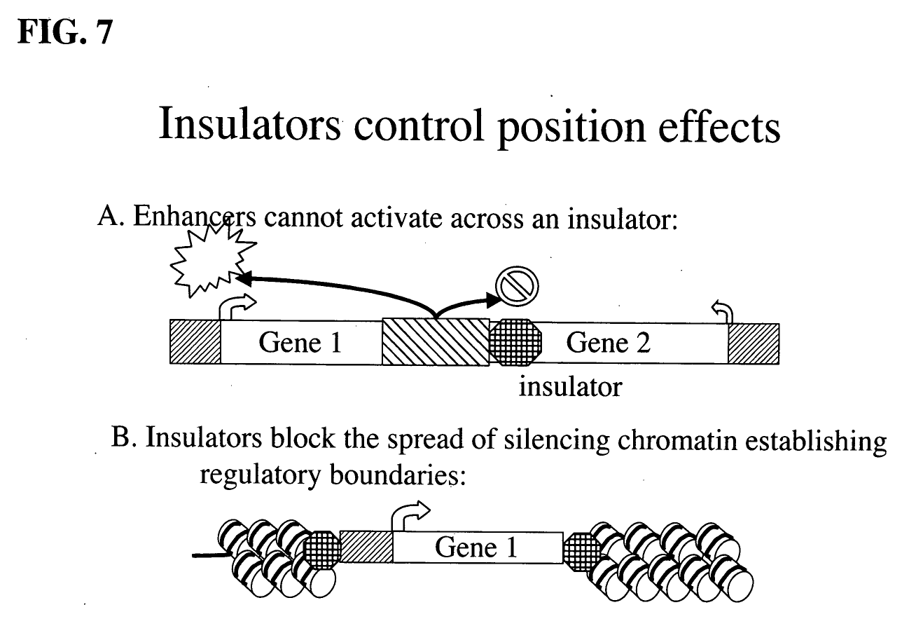

The chromosomal DNA of eukaryotic organisms is organized into a series of higher-order regions or “domains” that define discrete unites of compaction of chromatin. It is believed that the domain organization of eukaryotic chromatin may play a role in gene regulation. This phenomenon is referred to as “position effects.” Enhancer elements can activate gene relatively independently of position or orientation (see FIG. 6). Enhancers cannot activate across an insulator. Further, insulators ban block the spread of silencing chromatin establishing regulatory boundaries (see FIG. 7).

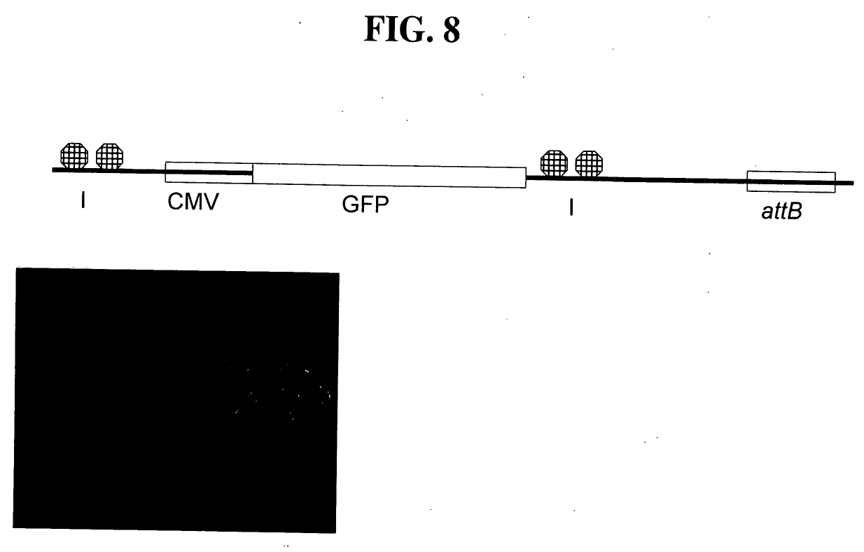

Insulators can relieve position effects on reporter sequences. The present inventors reconstructed a reporter plasmid establishing regulatory boundaries with tandem copies of insulators (from the chicken β-globin gene) flanking the CMV-GFP reporter and repeated the injections of plasmid with integrase mRNA into single-cell embryos. Uniform expression of GFP was achieved when the transgene was flanked with insulators (FIG. 8). The inventors also sought to determine if a tissue specific promoter from Xenopus laevis could be used in this procedure, and replaced the CMV promoter with a minimal ( 560 bp) promoter controlling GFP.

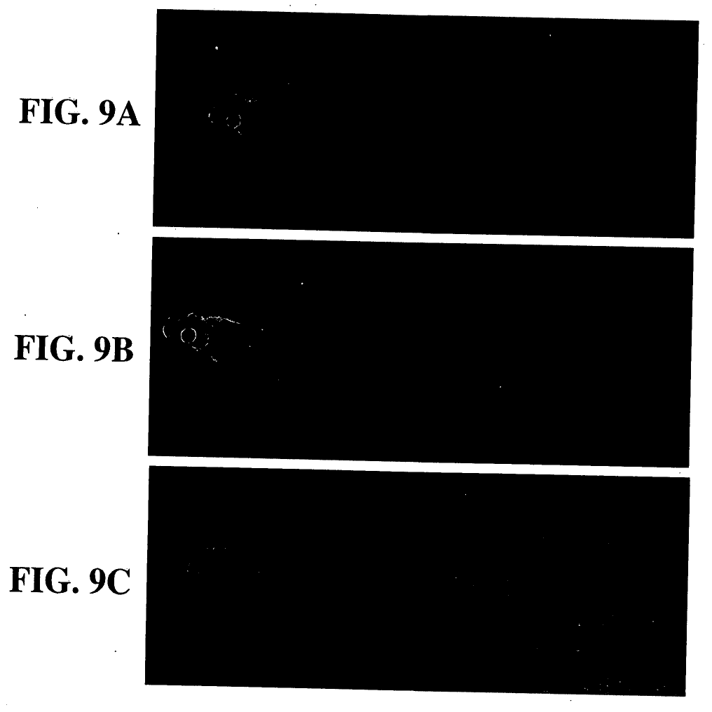

The inventors assayed the expression of CMV-GFP reporter plasmid when the transcription unit is bounded by insulator elements in tadpoles. In each panel of FIG. 9 is a brightfield and a fluorescent photograph of embryos treated at the single-cell stage and assayed at Stage 46. The tadpole injected with insulate CMV-GFP and integrase mRNA (panel C) fluoresced, whereas the others did not.

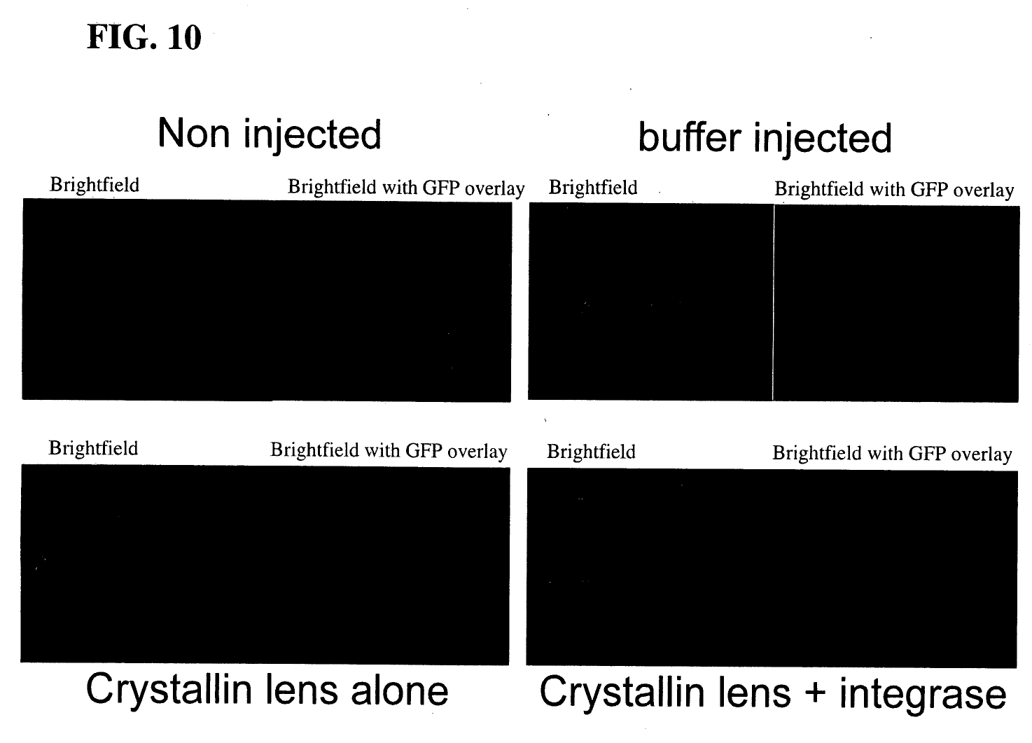

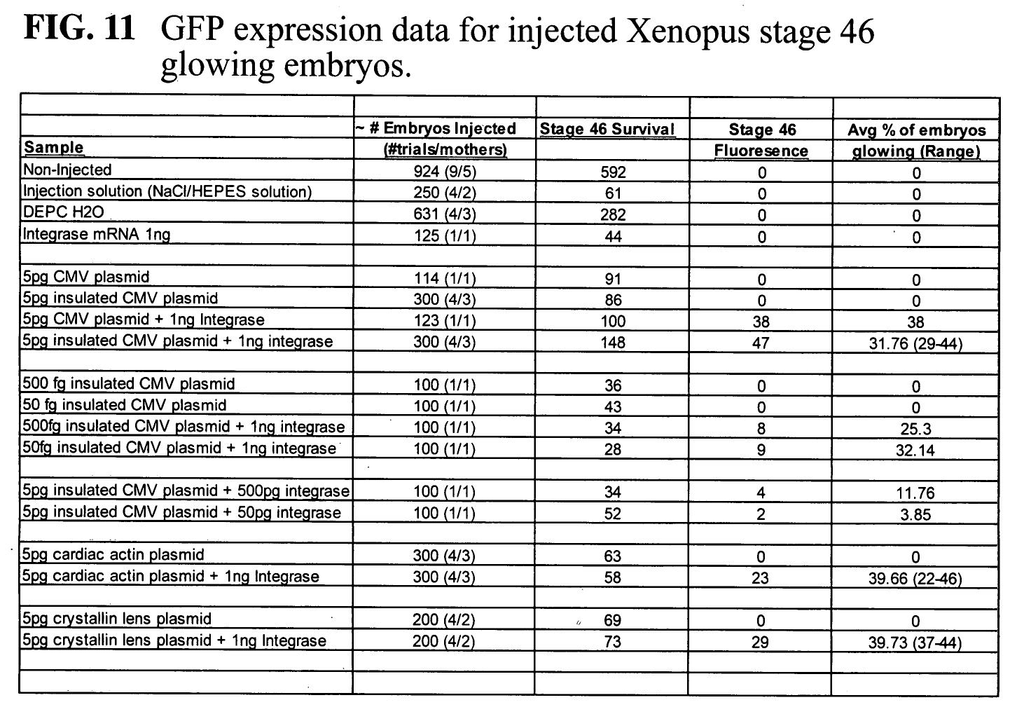

The inventors also generated tissue specific expression. When the CMV promoter was replaced with a minimal Xenopus crystallin lens promoter in the insulated GFP reporter construct, the construct induced lens-specific GFP expression (FIG. 10). FIG. 11 provides GFP expression data for injected Xenopus stage 46 glowing embryos.

In summary, phiC31 integrase can mediate integration of plasmid into the genome of developing Xenopus embryos efficiently. There are multiple integration sites that lead to position effect modulation of inserted gene expression. Further, position effects can be overcome using insulators to flank the integrated DNA.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A vector for site-specific integration of a polynucleotide sequence into an isolated eukaryotic cell's genome, the vector comprising:
(a) an isolated polynucleotide encoding a chromatin insulator element, wherein the insulator element when flanking a gene to be inserted into a host chromosome insulates the transcriptional expression of the gene from one or more cis-acting regulatory sequences in chromatin into which the gene has been inserted;
(b) a polynucleotide of interest operably linked to a eukaryotic promoter, and
(c) a single recombination site, wherein the single recombination site comprises a polynucleotide sequence that recombines with a second recombination site in the genome of the isolated eukaryotic cell and the recombination occurs in the presence of a site-specific recombinase.

2. The vector of claim 1, wherein the recombinase is a phiC31 phage recombinase, TP901-1 phage recombinase, or R4 phage recombinase.

3. The vector of claim 1, wherein the recombination site consists of a eukaryotic 5′ constitutive DNase I-hypersensitive site from the 5′ region of the chicken beta-globin gene locus, wherein insulator element is isolated from a 1.2 kilobase SacI-Sspl DNA fragment.

4. The vector of claim 1, wherein the insulator element is isolated from a higher eukaryotic organism.

5. The vector of claim 4, wherein the eukaryotic organism is a human.

6. The vector of claim 1, wherein the promoter is a tissue-specific promoter.

7. A method of site-specifically integrating a nucleic acid into a genome of a cell of a multicellular organism, the method comprising introducing the vector of claim 1 and a recombinase and/or a nucleic acid encoding a recombinase into a cell, and maintaining the cell under conditions sufficient for the recombination site to integrate into a genome attachment site in the genome of the cell by a recombination event mediated by the recombinase.

8. The method of claim 7, wherein the genome attachment site is a pre-selected site in the genome.

9. The method of claim 7, wherein the cell is a mammalian cell.

10. The method of claim 9, wherein the cell is a human cell.

11. The method of claim 7, wherein the vector comprises a tissue-specific promoter, resulting in tissue-specific expression of the target sequence.

12. A kit for use in integrating a nucleic acid into a genome of a cell of a multicellular organism, the kit comprising:
(a) a vector of claim 1; and
(b) a recombinase or nucleic acid encoding a recombinase.

13. The kit of claim 10, further comprising instructions for using the vector and the recombinase or nucleic acid encoding a recombinase in a method of modifying a genome of a cell of a multicellular organism.
